(19) United States
(12) Patent Application Publication
(10) Pub. No.: US 2015/0338397 A1
(43) Pub. Date: Nov. 26, 2015

(54) Use of Bufodienolides in Diagnosing and Treating Essential Hypertension

(71) Applicant: Jules B. Puschett, Houston, TX (US)

(72) Inventor: Jules B. Puschett, Houston, TX (US)

(21) Appl. No.: 14/720,886

(22) Filed: May 25, 2015

Related U.S. Application Data
(60) Provisional application No. 62/002,807, filed on May 24, 2014.

(51) Int. Cl. G01N 33/53; A61K 9/00; G01N 33/74; A61K 31/585

(52) U.S. Cl. CPC G01N 33/5308; A61K 31/585; A61K 9/0019; G01N 33/74; G01N 2333/575; G01N 2800/321; G01N 2800/50

(57) ABSTRACT

Certain embodiments are directed to methods of identifying patients with essential by detecting elevated levels of marinobufagenin (MBG) and treating the same with anti-MBG agents.

This application claims priority to U.S. Provisional Patent Application Ser. No. 62/002,807 filed May 24, 2014, which is incorporated herein by reference in its entirety.

### BACKGROUND

Hypertension is a major health problem, both in the United States and abroad. There are currently approximately 30 million Americans who suffer from this illness and many more times that number worldwide. The incidence of hypertension is more likely in subjects that are older, diabetic, and/or obese. Because the population is “aging,” and we are now experiencing epidemics of obesity and diabetes, we can expect hypertension to become even more prevalent in the years ahead.

There are etiopathogenetic groupings that help define the precise etiology of elevated blood pressure, i.e., volume expansion hypertension or vasoconstrictive hypertension. With regard to those patients who are likely to have the volume expansion variety of hypertension, there are 5 groups: (a) the elderly, (b) the obese, (c) African-Americans, (d) Hispanics, and (e) a subset of type II diabetics. In regard to patients who experience hypertension because of vasoconstrictive factors, the largest group includes patients with an excess of the major vasoconstrictor, angiotensin.

Currently, there is no test to determine if a patient has one or the other of these pathogenetic factors (or both) present as a major cause of the hypertension. It would be beneficial to have a method to distinguish between these two causes of hypertension.

### SUMMARY

Approximately 90-95% of all hypertensive patients can be categorized as having “essential” hypertension. However, this is not a single disease process, but one in which there are multiple etiologic factors, the phenotype for which is elevated blood pressure. The formula Q=P/R describes the relationship between blood flow (Q), blood pressure (P), and resistance to blood flow throughout the vascular tree (R). The formula solved for pressure is P=Q×R. Flow (Q) is measured as cardiac output (CO). Cardiac output represents the amount of blood flowing through the vascular circuit/minute. In the average human subject, this is approximately 6-8 L/minute. The largest influence on CO is vascular volume, which is a reflection of the amount of salt and water in the body. Extracellular fluid volume (ECFV), which includes the plasma volume, is a reflection of vascular volume. These considerations provide us with a mechanism to divide essential hypertension into its overall etiologic categories: (1) Patients with expanded ECFV, that is, volume expanded patients with increased total body salt and water; and (2) vasoconstrictive factors which raise vascular resistance.

Certain embodiments are directed to methods for determining if a patient has volume expansion and/or a vasoconstrictive pathogenesis as the primary etiology. In certain aspects the methods include measuring body volume and vasoconstriction, or representative biomarkers to classify a hypertensive subject. The methods can provide a physician with the knowledge/ability to select the correct drug to use on a patient at the first patient visit. This shortens the time needed to control the patient's blood pressure. Thus, if the patient has volume expansion-mediated hypertension, appropriate therapy directed at resolving this problem is instituted. If the patient has vasoconstrictive hypertension, an agent directed at resolving this problem is prescribed.

As used herein, the term “analyte” generally refers to a substance to be detected. For instance, analytes may include antigenic substances such as small molecules or proteins. Analytes include, but are not limited to, organic compounds, proteins, and peptides. Specific examples of some analytes include MBG and angiotensinogen.

As used herein, the term “test sample” or “sample” generally refers to a biological material suspected of containing an analyte of interest. The test sample may be derived from a biological source, such as a physiological fluid, including, blood or urine. The test sample may be used directly as obtained from the biological source or following a pretreatment to modify the character of the sample. For example, such pretreatment may include preparing plasma from blood and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Mean systolic blood pressure (BP) values in sham (SHM) animals, angiotensin-infused rats (ANG), angiotensin infused animals given resibufogenin (ANGR), normal rats rendered hypertensive by the infusion of saline (volume expansion induced hypertensive rats) (NDS), NDS rats given resibufogenin (NDSR). While both angiotensin infusion and saline volume expansion raised blood pressure equivalently, resibufogenin reduced blood pressure only in the volume expanded animals.

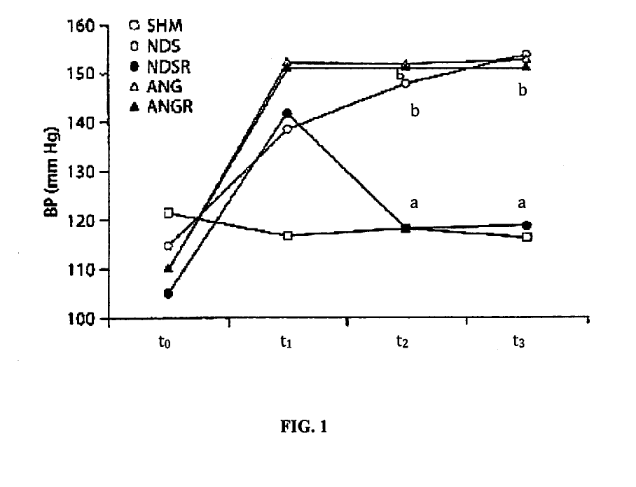

### DESCRIPTION

Bufodienolides were discovered in amphibians and extracted from plants. They are thought to act by virtue of their ability to inhibit Na+/K+-ATPase activity (Flier et al., Science 1980, 208:503-05). Several bufodienolides have been suggested as candidate sodium pump ligands (SPLs) in mammals, including marinobufagenin (MBG), which acts in vitro as a vasoconstrictor (Fedorova et al., Am. J. Hypertens. 1997, 10:929-35; Lopatin et al., J. Hypertens. 1999, 17:1179-87). Enhanced MBG production occurs in pathological states associated with fluid retention, including essential and salt-sensitive hypertension, preeclampsia, and uremic cardiomyopathy (Gonick et al, Clin. Exp. Hypertens. 1998, 20: 617-27, Bagrov et al., Hypertension 1998, 31:1097-1103; Fedorova et al., Hypertension 2001, 37:462-66; Lopatin et al., J. Hypertens. 1999, 17:1179-87; Fedorova et al, Circulation 2002; 105: 1122-27; Kennedy et al., Hypertension 2006, 47:448-495).

Bufodienolides (Formulas I, II, and III below) and related substances called the cardenolides (Formulas IV, V, and VI below) are termed the “cardiotonic steroids” or “cardiac glycosides.” They are similar in general structure but different in specific structure and function. However, all of the cardiotonic steroids have the ability to inhibit the ubiquitous enzyme sodium/potassium ATPase (Na+/K+ATPase).

Certain aspects described herein relate in part to marinobufagenin (MBG), the most extensively studied of the bufodienolides. MBG is elevated in disturbances that result from excessive volume expansion (Gonick et al. Clin. Exp. Hypertens. 20: 617-627, 1998; Ianosi-Irimie et al. Clin. Exp. Hypertens. 8: 605-617, 2005; Vu et al. Exp. Biol. Med. 231: 215-220, 2006). An immunoassay has been developed to measure MBG in both serum and urine (Abi-Ghanem et al. Journal of Immunoassay and Immunochemistry 32: 31-46, 2011). Certain embodiments are directed to the measurement of MBG for determining whether or not excessive volume expansion exists.

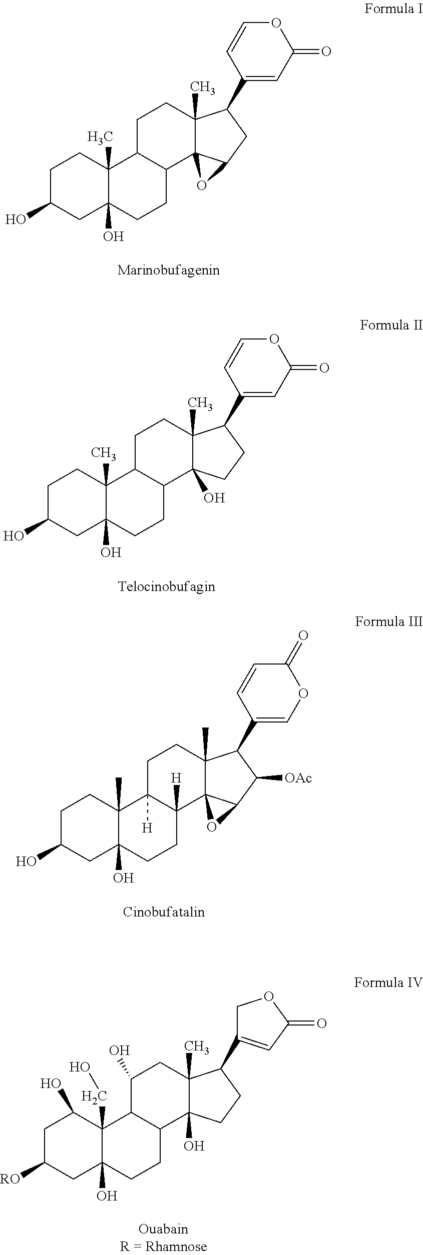

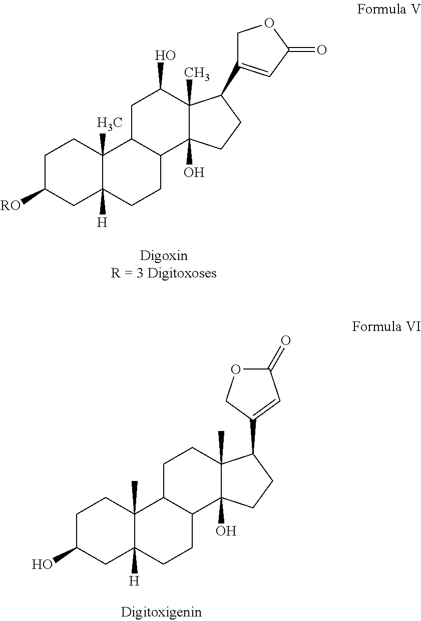

Angiotensin is the major vasoconstrictor in the human body. The activity of the renin-angiotensin system can be evaluated by measurement of the urinary concentration of angiotensinogen (Kobori et al. Int Rev Thromb 6: 108-116, 2011). In certain aspects the concentration or levels of MBG and/or angiotensinogen can be determined and the pathogenesis of hypertension determined in a patient based on these levels. Accordingly, appropriate therapy for either or both of these abnormalities is prescribed based on the resulting levels of MBG and angiotensinogen.

MBG is elevated in other syndromes such as preeclampsia and traumatic brain injury. It has been shown that the administration of the antagonist of MBG, resibufagenin (RBG) is therapeutically effective. RBG administration results in an amelioration of tissue injury in a rodent brain contusion model when given 1 hour after the insult and prevents preeclampsia in animal models. RBG antagonizes MBG activity and may be an effective treatment for hypertension, ARDS, traumatic brain injury, and preeclampsia. Certain embodiments are directed to treating a hypertensive patient having elevated levels of MBG by administering an MBG antagonist or anti-MBG agent. In certain aspects the anti-MBG agent is resibufagenin (RBG).

### I. Methods for Classifying Hypertensive Patients

Essential hypertension is a term that refers to patients with high blood pressure who do not have secondary causes (e.g., kidney failure or insufficiency, pheochromocytoma, primary hyperaldosteronism, etc.) and accounts for 90-95% of all patients presenting for evaluation of elevated blood pressure. Patients with essential hypertension can be subcategorized as to whether the disorder is primarily related to volume expansion (too much salt and water in the body) or to vasoconstriction. The ability to determine the etiology of hypertension in an outpatient (or an inpatient) setting when a patient is first identified as hypertensive can provide a more rapid and accurate assignment of that patient to a therapy most likely to be effective in that particular patient. This would replace current therapeutic guesswork and would result in a diagnostic/therapeutic “match,” an example of “personalized medicine.” Accordingly, patients can be categorized as having volume expansion-mediated or vasoconstrictive hypertension or both.

Hypertension currently affects about 30% of the US population and many times that number worldwide. While the current approximation of the proportions of patients who have the volume expansion variety of hypertension is approximately 30-40% of the hypertensive population, while the group suffering from vasoconstriction hypertension is about 60-70%. Those figures are rapidly changing because patients who have a propensity to develop the volume expansion-mediated form of elevated blood pressure include: (1) the elderly, (2) the obese, (3) African-Americans (4) Hispanics and (5) a subset of type II diabetics. Notably, our population is aging, there is an epidemic of obesity and diabetes in the United States, and African-Americans and Hispanics are an increasing percentage of the population. Thus, it is postulated that in the next 20-30 years, the larger percentage of patients with the volume expansion type of hypertension will exceed those with the vasoconstrictive form, reversing the current proportions.

There are currently limited diagnostic methods for determining which patient has volume expansion and which has a vasoconstrictive basis for his/her hypertension. Described herein is a rapid, easily performed test of hypertension so that the correct therapeutic agent is administered/prescribed when the correct etiologic diagnosis is determined at the patient's first visit. This determination is made on the following bases:

Evidence has accumulated that indicates that the level of MBG in the blood and/or urine is a reflection of body volume (Puschett et al., Biochimica et Biophysica Acta 1802:1246-53, 2010; Lichardus and Pearce, Nature 5021:407-409, 1966). In certain aspects an elevation in the amount of MBG is indicative of the patient suffering from an expansion of his body fluid volume. Thus, the patient having elevated levels of MBG will be administered an agent directed toward reducing body volume (e.g., a diuretic) or an agent that antagonizes MBG. In certain aspects an MBG antagonist is resibufogenin (RBG).

With regard to vasoconstriction, the major agent found to subserve this function in the body's vasculature is angiotensin, which is produced by the kidney (Reviewed in Kobori et al., Pharmacological Reviews 59:251-87, 2007). Angiotensin is metabolized and produces a metabolite called angiotensinogen. The levels of angiotensinogen are indicative of the activity of the renin-angiotensin system in the body. The activity of the renin-angiotensin system in the body can be measured by determining the amount of angiotensinogen excreted in the urine. Accordingly, a simplified, reproducible test for the simultaneous determination of MBG and angiotensinogen can be utilized to determine which form of hypertension exists in the individual patient by detecting MBG and angiotensinogen in a sample from a patient, allowing the appropriate treatment to be prescribed based upon categorization of a patient. A test kit can be provided and made available in the physician's office, in the clinic, in the field, or in a hospital laboratory.

In certain aspects a patient is tested and is positive for elevated levels of both MBG and angiotensinogen, then the patient is administered an anti-hypertensive therapy that includes agents for treating volume expansion hypertension and vasoconstrictive hypertension. In the case where MBG is elevated and angiotensinogen is not elevated then a patient is administered a therapeutic agent(s) to treat the volume expansion form of hypertension. In the event that MBG levels are not elevated and angiotensinogen levels are elevated then a patient is administered a therapeutic agent(s) to treat vasoconstrictive hypertension. A non-limiting example of a therapy for vasoconstrictive hypertension includes administration of an angiotensin converting enzyme (ACE) inhibitor and/or calcium channel blocker. A non-limiting example of a volume expansion hypertension therapy includes administration of a diuretic and/or a MBG antagonist.

In certain aspects, elevated levels of MBG or angiotensinogen are at least 30, 40, 50, 60, 70, 80, 90, 100, 200% or more than normal levels or a reference level. In other aspects elevated MBG or angiotensinogen levels are at least 1.5, 2, 3, or more times the normal levels or a reference level. In certain embodiments MBG and angiotensinogen are detected or measured in blood or urine.

II. Methods of Detecting Marinobufagenin (MBG) and/or Angiotensinogen

MBG and/or angiotensinogen can be detected using a variety of assays including, but not limited to immuno-detection, microchip, or lateral flow based methods. Immuno-based assays include, but are not limited to radiolabeled, enzyme, fluorescence, dot blot, chemiluminescence, dip-stick, or biosensor assays. See, for example, Principles and Practice of Immunoassays, Price, C. P. and Newman, D. J. (Eds.), Stockton Press, 1997; The Immunoassay Handbook; 2nd Edition, Wild, D. (Ed.), Nature Publishing Group, London, 2001.

Methods for measuring MBG in the blood or urine can provide sensitivities in the pg/ml (picograms per milliliter) range or pg/mg creatinine, respectively. In certain aspects the MBG detection methods employ an MBG specific ELISA assay. In certain aspects the MBG assay discriminates between MBG and resibufogenin (RBG). In certain aspects, antibodies are used to detect the presence of MBG in an original or processed sample obtained from a subject (Abi-Ghanem et al., Journal of Immunoassay and Immunochemistry, 32:31-46, 2011). Samples obtained from a subject may include, for example, cells, tissue, blood, serum, or urine. For example, a sample can be blood or urine collected from a subject. A sample can be analyzed directly or extracted before analysis.

Methods for measuring angiotensinogen in the blood or urine can provide sensitivities in the nmol/ml (nanomole per milliliter) range or μg/g creatinine, respectively. In certain aspects the angiotensinogen detection methods employ an angiotensinogen specific ELISA assay. In certain aspects, antibodies are used to detect the presence of angiotensinogen in an original or processed sample obtained from a subject. Samples obtained from a subject may include, for example, cells, tissue, blood, serum, or urine. For example, a sample can be blood or urine collected from a subject. A sample can be analyzed directly or extracted before analysis.

In certain aspects a sample is contacted with an effective amount of one or more antibody/antibodies and the sample is screened to detect an antibody-MBG (e.g., US 20100158900) or antibody-angiotensinogen (e.g., 27412A Human Total Angiotensinogen Assay Kit, Clontech) complex, such as detecting the binding reaction between the antibody and the MBG or the angiotensinogen. Detection of an antibody-antigen complex or binding reaction indicates that the sample contains MBG or angiotensinogen. In some embodiments, the one or more antibody is labeled with a detectable moiety, such as a fluorescent label, so that its signal changes upon binding to MBG or to angiotensinogen.

In other embodiments, MBG or angiotensinogen in the sample is immobilized on a surface and detected. In certain aspects MBG or angiotensinogen is immobilized prior to introduction of the labeled antibody, and the amount of the signal, corresponding to the amount of bound labeled antibody, correlates to the amount of MBG or angiotensinogen in the sample. In still other embodiments, MBG or angiotensinogen is captured by an immobilized unlabeled first antibody, after which a labeled second antibody is introduced to bind to the captured MBG or angiotensinogen and produce a signal in proportion to the amount of captured MBG or angiotensinogen. In other embodiments the binding of MBG or angiotensinogen is detected by altering the properties of the surface to which it is directly or indirectly bound.

In general, an antibody can be used as a labeled primary reagent in a direct assay or as an unlabeled reagent to be detected by a secondary developing antibody conjugate, such as labeled anti-rabbit antibody, in an indirect assay (Abi-Ghanem et al., Journal of Immunoassay and Immunochemistry, 32:31-46, 2011). Additionally, an antibody can be used in a competition assay to detect an antigen or antibody in a sample. For example, MBG or angiotensinogen in a sample extract is captured by an unlabeled antibody immobilized on the surface of an ELISA well and then detected by a labeled antibody of the same or different kind and/or specificity. Alternatively, the sample can be suspended in a buffer and mixed directly with an antibody, thus allowing the antibody to form an immune complex with MBG or angiotensinogen. The reduction of free antibody due to complex formation can then be determined in a second step, based on solid-phase ELISA with purified MBG or angiotensinogen, by comparing the relative reactivity of free residual antibody left over after sample incubation (sample reactivity) to that of the same antibody when not mixed with the sample (reference reactivity). The ratio of sample to reference antibody reactivity will be inversely proportional to the amount of MBG or angiotensinogen in the sample.

In certain aspects, methods of the invention can be adapted for lateral flow assays and devices supporting such. Lateral flow assays, also known as immunochromatographic assays, are typically carried out using a simple device intended to detect the presence (or absence) of a target analyte in the sample. Most commonly these tests are used for medical diagnostics either for home testing, point of care testing, or laboratory use. Often produced in a dipstick format, these assays are a form of immunoassay in which the test sample flows along a solid substrate via capillary action. After the sample is applied to the test it encounters a colored or labeling reagent which mixes with the sample and transits the substrate encountering lines or zones which have been pretreated with an antibody or antigen or affinity reagent. Depending upon the analyte present in the sample the colored or labeling reagent can become bound at the test line or zone. Lateral flow assays can operate as either competitive or sandwich assays.

In still a further aspect, methods of the invention can be adapted for protein array assays and devices supporting such. Protein arrays or microarrays (also known as a biochip, or a protein chip) are measurement devices used in biomedical applications to determine the presence and/or amount of an analyte(s) in biological samples, e.g. blood, urine, swabs, tissues scrappings, etc. Typically, a number of different capture agents, most frequently monoclonal antibodies, can be deposited on a chip surface (glass or silicon) in an array. This format is also referred to as a microarray (a more general term for chip based biological measurement devices).

In yet another aspect the assay can be a microfluidic chip-based assay. Microfluidics is an important innovation in biochip technology. Since microfluidic chips can be combined with immunodetection and mass spectrometric analysis (See Wang et al., Lab Chip 13:4190-97, 2013; Baker et al. Bioanalysis 1(5): 967-75 2009; Wang et al., Anal. Chem. 72:832-839, 2000).

### III. Anti-Hypertensive Therapy

In certain embodiments, the invention also provides compositions comprising an anti-MBG agent or MBG antagonist with one or more of the following: a pharmaceutically acceptable diluent, a carrier, a solubilizer, an emulsifier, and/or a preservative. Such compositions may contain an effective amount of at least one anti-MBG agent. The use of an anti-MBG agent in combination with other anti-hypertensive therapy is also included.

In one aspect an anti-MBG agent is resibufagenin (RBG; epoxy-3β-hydroxy-5β-bufa-20,22-dienolide) or a functional analog thereof. Resibufagenin has the structure shown in Formula VII. Other MBG antagonists can be produced that maintain the structural context of the hydrogen at the 5 position of resibufagenin.

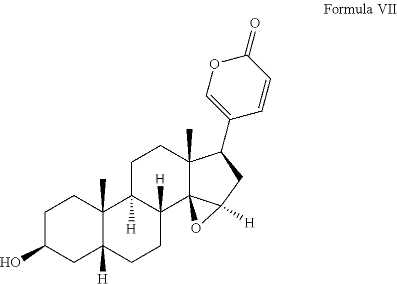

This compound differs from MBG only in the absence of a hydroxyl group in the beta-5 position of the molecule. When administered to experimental animals, RBG resolves blood pressure elevations resulting from volume expansion-mediated hypertension and does not lower elevated blood pressure in animals subjected to angiotensin excess (Danchuk et al. Am J Nephrol 28: 8-13, 2008). Current therapy of volume expansion hypertension involves the administration of a diuretic. RBG can be considered in place of the diuretic as the therapy of choice of volume expansion-mediated hypertension or can be utilized in combination with the diuretic so that smaller dosages of each agent could be utilized. The latter strategy can result in fewer side effects from either drug.

Treatment of vasoconstrictive hypertension can be accomplished by administration of the angiotensin converting enzyme (ACE) inhibitors or an angiotensin receptor blocker (ARB) agent.

The anti-hypertensive agents may be formulated into therapeutic compositions in a variety of dosage forms such as, but not limited to, liquid solutions or suspensions, tablets, pills, powders, suppositories, polymeric microcapsules or microvesicles, liposomes, and injectable or infusible solutions. The preferred form depends upon the mode of administration and the particular disease targeted. The compositions also preferably include pharmaceutically acceptable vehicles, or carriers, well known in the art.

Acceptable formulation components for pharmaceutical preparations are nontoxic to recipients at the dosages and concentrations employed. In addition to the anti-hypertensive agents that are provided, compositions may contain components for modifying, maintaining, or preserving, for example, the pH, osmolarity, viscosity, clarity, color, isotonicity, odor, sterility, stability, rate of dissolution or release, adsorption, or penetration of the composition. Suitable materials for formulating pharmaceutical compositions include, but are not limited to, amino acids (such as glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophobic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (see Remington's Pharmaceutical Sciences, 18 th Ed., (A. R. Gennaro, ed.), 1990, Mack Publishing Company), hereby incorporated by reference.

Formulation components are present in concentrations that are acceptable to the site of administration. Buffers are advantageously used to maintain the composition at physiological pH or at a slightly lower pH, typically within a pH range of from about 4.0 to about 8.5, or alternatively, between about 5.0 to 8.0. Pharmaceutical compositions can comprise TRIS buffer of about pH 6.5-8.5, or acetate buffer of about pH 4.0-5.5, which may further include sorbitol or a suitable substitute therefor.

The pharmaceutical composition to be used for in vivo administration is typically sterile. Sterilization may be accomplished by filtration through sterile filtration membranes. If the composition is lyophilized, sterilization may be conducted either prior to or following lyophilization and reconstitution. The composition for parenteral administration may be stored in lyophilized form or in a solution. In certain embodiments, parenteral compositions are placed into a container having a sterile access port, for example, an intravenous solution bag or vial having a stopper pierceable by a hypodermic injection needle, or a sterile pre-filled syringe ready to use for injection.

The above compositions can be administered using conventional modes of delivery including, but not limited to, intravenous, intraperitoneal, oral, intraarterial, and intrapleural. In certain aspects an anti-hypertensive agent will be administered orally. When administering the compositions by injection, the administration may be by continuous infusion or by single or multiple boluses. For parenteral administration, the anti-hypertensive agents may be administered in a pyrogen-free, parenterally acceptable solution comprising the desired anti-hypertensive agents in a pharmaceutically acceptable vehicle. A particularly suitable vehicle for parenteral injection is one in which one or more anti-MBG and/or anti-angiotensin agents are formulated as a sterile solution, properly preserved.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

If desired, stabilizers that are conventionally employed in pharmaceutical compositions, such as DMSO, oil, sucrose, trehalose, or glycine, may be used. Typically, such stabilizers will be added in minor amounts ranging from, for example, about 0.1% to about 0.5% (w/v). Surfactant stabilizers, such as TWEEN®-20 or TWEEN®-80 (ICI Americas, Inc., Bridgewater, N.J., USA), may also be added in conventional amounts. In certain aspects the composition are 10 to 30% DMSO and/or oil (e.g., sesame oil).

The components used to formulate the pharmaceutical compositions are preferably of high purity and are substantially free of potentially harmful contaminants (e.g., at least National Food (NF) grade, generally at least analytical grade, and more typically at least pharmaceutical grade). Compositions for parental administration are also sterile, substantially isotonic and made under GMP conditions.

For the compounds of the present invention, alone or as part of a pharmaceutical composition, such doses are between about 0.001 mg/kg and 1 mg/kg body weight, preferably between about 1 and 100 μg/kg body weight, most preferably between 1 and 10 μg/kg body weight.

Therapeutically effective doses will be easily determined by one of skill in the art and will depend on the severity and course of the disease, the patient's health and response to treatment, the patient's age, weight, height, sex, previous medical history and the judgment of the treating physician.

1. A method for sub-classifying patients with essential hypertension comprising (a) contacting a patient sample with a substrate having a first binding reagent that specifically binds marinobufagenin (MBG) and a second binding reagent that specifically binds angiotensinogen; (b) detecting and quantitating MBG-binding reagent complexes and angiotensinogen-binding reagent complexes; and (c) classifying a patient as having (i) volume expansion hypertension if MBG levels are elevated and angiotensinogen levels are not elevated, (ii) vasoconstrictive hypertension if angiotensinogen levels are elevated and MBG levels are not elevated, or (iii) both volume expansion hypertension and vasoconstrictive hypertension if MBG levels are elevated and angiotensinogen levels are elevated.

2. A method of identifying a patient at risk of developing volume expansion-mediated hypertension comprising determining the marinobufagenin (MBG) level in blood or urine of a patient having hypertension or is at risk of developing hypertension; and identifying the patient as in need for volume expansion-mediated hypertension therapy when a blood level of MBG is at least 50% greater than a reference level or a urine level is at least 50% greater than a reference level.

3. The method of claim 1, further comprising administering a volume expansion-mediated hypertension treatment to a subject having elevated levels of MBG.

4. The method of claim 1, wherein the MBG level is measured using an ELISA assay.

5. The method of claim 1, wherein the MBG level is measured using a lateral flow assay.

6. The method of claim 1, wherein an elevated blood level of MBG is greater than 45 pg/ml or elevated urine level is greater than 450 pg MBG/mg creatinine.

7. The method of claim 1, wherein an at risk subject is diagnosed with volume expansion pathogenesis or vasoconstrictive pathogenesis.

8. The method of claim 6, wherein the level of angiotensinogen is measured to determine diagnosis of volume expansion pathogenesis or vasoconstrictive pathogenesis.

9. A method of treating a patient having volume expansion-mediated hypertension comprising administering to the patient an effective amount of marinobufagenin (MBG) antagonist.

10. The method of claim 8, wherein the MBG antagonist is resibufagenin (RBG).

11. The method of claim 8, wherein the MBG antagonist is administered intravenously.

12. The method of claim 8, wherein a diuretic is not administered.
